(19) United States
(12) Patent Application Publication
(10) Pub. No.: US 2019/0169100 A1
(43) Pub. Date: Jun. 6, 2019

(54) PROCESS FOR THE DEHYDROCHLORINATION OF A CHLOROALKANE

(71) Applicant: BLUE CUBE IP LLC, Clayton, MO (US)

(72) Inventors: John D. Myers, Clayton, MO (US); Max Tirtowidjojo, Clayton, MO (US)

(73) Assignee: BLUE CUBE IP LLC, Clayton, MO (US)

(21) Appl. No.: 16/311,761

(22) PCT Filed: Jun. 22, 2017
(86) PCT No.: PCT/US2017/038792

§ 371 Date: Dec. 20, 2018

Related U.S. Application Data
(60) Provisional application No. 62/353,860, filed on Jun. 23, 2016.

(51) Int. Cl. C07C 17/25; C07C 17/04; B01J 27/10

(52) U.S. Cl. CPC C07C 17/25; B01J 27/10; C07C 17/04

(57) ABSTRACT

A process for the production of a chloroalkene that includes contacting a liquid phase chloroalkane with a solid catalyst, a catalyst deposited on a solid substrate, or combinations thereof, thereby causing the dehydrochlorination of the chloroalkane to produce a chloroalkene product and HCl. Processes for the production of 1,1,2,3-tetrachloropropene and 1,1,1,2,3-pentachloropropane are further provided.

### FIELD

The present disclosure relates to processes for the production of chloroalkenes. In particular, the present disclosure relates to catalytic dehydrochlorination of chloroalkanes using a solid catalyst, a catalyst deposited on a solid substrate, or combinations thereof.

### BACKGROUND

Chlorocarbons, in particular, chlorinated propenes are used as feedstocks for the manufacture of hydrofluoroolefin (HFO) products, polyurethane blowing agents, biocides, and polymers, among other chemical products. However, many chlorinated propenes have limited commercial availability or are available only at a prohibitively high cost due in part to the multi-step processes typically utilized in their manufacture. For example, conventional processes may involve multiple chlorination and dehydrochlorination steps to arrive at a desired level of chlorination in the final product. The dehydrochlorination of chlorocarbons may be carried out using dissolved metal chloride catalysts, requiring subsequent distillation or separation of the chemical product from the catalyst. The presence of such dissolved catalytic components can produce further undesired reactions prior to and during the distillation process. It would thus be desirable to provide improved processes for the production of chlorocarbons useful as feedstocks in the synthesis of commercial chemical products.

### SUMMARY

The present disclosure provides a process for the production of a chloroalkene. The process includes contacting a liquid phase first chloroalkane with a solid catalyst, a catalyst deposited on a solid substrate, or combinations thereof, thereby causing dehydrochlorination of the first chloroalkane to produce a first chloroalkene product and HCl.

### BRIEF DESCRIPTION OF THE DRAWINGS

In order to describe the manner in which the advantages and features of the disclosure can be obtained, reference is made to embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIG. 1 shows the percentage conversion of 1,1,1,3-tetrachloropropane versus temperature after being contacted with 5.6 g of 30 weight percent ZnCl₂ catalyst on a carbon substrate, at a flow rate of 0.33 cc/min and at a pressure of 170 psig;

FIG. 2 shows the percentage trichloropropene selectivity versus temperature after 1,1,1,3-tetrachloropropane was contacted with 5.6 g 30 weight percent ZnCl₂ catalyst on a carbon substrate, at a flow rate of 0.33 cc/min and at a pressure of 170 psig;

FIG. 3 shows the percentage conversion of 1,1,1,3-tetrachloropropane versus temperature after being contacted with a 5 g y-type molecular sieve as catalyst, at a flow rate of 0.33 cc/min and at a pressure of 170 psig, with and without simultaneous chlorination of the trichloropropene product by sulfuryl chloride;

FIG. 4 shows the percentage trichloropropene selectivity versus temperature after 1,1,1,3-tetrachloropropane was contacted with a 5 g y-type molecular sieve as catalyst, at a flow rate of 0.33 cc/min and at a pressure of 170 psig, with and without simultaneous chlorination of the trichloropropene product by sulfuryl chloride;

FIG. 5 shows the percent conversion of 1,1,1,3-tetrachloropropane, percent trichloropropene selectivity, and production rate in g/H versus time in the presence of 5 g y-type molecular sieve as catalyst, at a flow rate of 0.33 cc/min and at a pressure of 170 psig, with and without simultaneous chlorination of the trichloropropene product by sulfuryl chloride;

FIG. 6 shows the percentage conversion of 1,1,1,3-tetrachloropropane, percentage trichloropropene selectivity, feed rate in g/H, and temperature versus time in the presence of 4.9 g 13× molecular sieve in the form of 16×40 mesh beads, at a flow rate of 0.33 cc/min and at a pressure of 170 psig; and

FIG. 7 shows the percentage conversion of 1,1,1,3-tetrachloropropane, percentage trichloropropene selectivity, feed rate in g/H, and temperature versus time in the presence of 4.2 g 22.9 percent by weight FeCl₃ catalyst on activated carbon, at a flow rate of 0.33 cc/min and at a pressure of 100 psig.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

It should be understood at the outset that although illustrative implementations of one or more embodiments are illustrated below, the disclosed processes may be implemented using any number of techniques. The disclosure should in no way be limited to the illustrative implementations and techniques described herein, but may be modified within the scope of the appended claims along with their full scope of equivalents.

In the following discussion and in the claims, the terms “including” and “comprising” are used in an open-ended fashion, and thus should be interpreted to mean “including, but not limited to . . . ”. The terms “first,” “second,” and the like, as used herein, do not denote any quantity, or importance, but rather are used to distinguish one element from another. Also, the terms “a” and “an” do not denote a limitation of quantity, but rather denote the presence of at least one of the referenced item. As used herein, percent (%) conversion is meant to indicate change in molar or mass flow of reactant in a reactor in ratio to the incoming flow, while percent (%) selectivity means the change in molar flow rate of product in a reactor in ratio to the change of molar flow rate of a reactant. If ranges are disclosed, the endpoints of all ranges directed to the same component or property are inclusive and independently combinable (e.g., the range of “5 wt. % to 25 wt. %,” is inclusive of the endpoints and intermediate values of the ranges of “5 wt. % to 25 wt. %,” etc.). The various characteristics described in more detail below, will be readily apparent to those skilled in the art with the aid of this disclosure upon reading the following detailed description, and by referring to the accompanying drawings.

The present disclosure generally relates to a process for the production of a chloroalkene by catalytic dehydrochlorination of a chloroalkane using a solid catalyst or a catalyst deposited on a solid support. According to at least one aspect of the present disclosure, the process may include contacting a liquid phase first chloroalkane with a solid catalyst, a catalyst deposited on a solid substrate, or combinations thereof, thereby causing dehydrochlorination of the first chloroalkane to produce a first chloroalkene product and HCl. The catalyst may include a metal chloride deposited on a solid substrate. Suitable metal chlorides may include, but are not limited to, ZnCl₂, FeCl₃, AlCl₃, CuCl₂, and combinations thereof. In at least some instances, the catalyst may include more than one metal chloride, including, but not limited to, metal chlorides having different metal oxidation states. For example, the catalyst may include a mixture of ZnCl₂ and FeCl₃ or a mixture of FeCl₂ and FeCl₃. In at least some instances, the catalyst may include a reduced (zero-valence) metal, such as Cu or Fe.

Suitable solid substrates may include, but are not limited to, substrates comprising carbon, alumina, silica, titania, an aluminosilicate, a zeolite, and combinations thereof. In at least some instances, the solid substrate may include a molecular sieve, including, but not limited to, an aluminosilicate molecular sieve, a zeolite molecular sieve, a Y-type molecular sieve, a 13× molecular sieve, and combinations thereof.

The metal chloride or reduced metal catalysts may be used in any combination with any suitable solid substrate without departing from the spirit and scope of the present disclosure. For example, ZnCl₂, FeCl₃, CuCl₂, or any combination thereof, may be deposited on a carbon substrate or may be deposited on a different solid substrate, such as alumina, silica, titania, an aluminosilicate, a zeolite, or combinations thereof.

According to at least one aspect of the present disclosure, the presently disclosed process may further include reacting at least a portion of the first chloroalkene product with a chlorinating agent to form at least some of a second chloroalkane product. In at least some instances, the first chloroalkane and the second chloroalkane product are chemically different. Suitable chlorinating agents may include, but are not limited to, chlorine gas, liquid chlorine, sulfuryl chloride, and combinations thereof. The chlorinating agent is essentially dry, i.e., it has a water content of the below 1000 ppm. Lower water concentrations are preferred, but not required. In at least some instances, the dehydrochlorination of the first chloroalkane and the chlorination of the first chloroalkene product may occur contemporaneously, or nearly contemporaneously. For example, the first chloroalkene product may be chlorinated as soon as the first chloroalkene product is produced by the dehydrochlorination of the first chloroalkane. In at least some instances, the dehydrochlorination of the first chloroalkane and the chlorination of the first chloroalkene product occurs contemporaneously in the same vessel, in different sections of the same vessel, or in separate vessels.

The dehydrochlorination of the first chloroalkane may be conducted at a temperature of from about 0° C. to about 200° C. In at least some instances, the dehydrochlorination of the first chloroalkane may be conducted at a temperature of from about 25° C. to about 170° C. In other cases, a suitable temperature for the dehydrochlorination of the first chloroalkane may be from about 80° C. to about 150° C.

In some cases, the dehydrochlorination of the first chloroalkane may desirably occur at temperatures between 25° C. and 170° C., or between 50° C. and 170° C., or between 75° C. and 170° C., or between 100° C. and 170° C., or between 125° C. and 170° C., or between 150° C. and 170° C. In other cases, the dehydrochlorination of the first chloroalkane may desirably occur at temperatures between 80° C. and 150° C., or between 90° C. and 150° C., or between 100° C. and 150° C., or between 110° C. and 150° C., or between 120° C. and 150° C., or between 130° C. and 150° C., or between 140° C. and 150° C.

The dehydrochlorination of the first chloroalkane may be further conducted at a pressure of from about 0 psig to about 200 psig. In general, the pressure must be high enough to ensure that at least some of the first chloroalkane is in liquid phase during the dehydrochlorination reaction. For example, if the first chloroalkane is 1,1,1,3-tetrachloropropane, the dehydrochlorination of 1,1,1,3-tetrachloropropane may be conducted at the minimum pressure required to ensure that most of the 1,1,1,3-tetrachloropropane is in liquid phase at the selected temperature of reaction. In at least some instances, the dehydrochlorination of the first chloroalkane may be conducted at a pressure and temperature in which the first or second chloroalkane is in liquid phase. Liquid feed rates during the dehydrochlorination of the first or second chloroalkane may be from about 0.001 to about 1 cc/min/g catalyst.

If desired, the presently disclosed process may further include additional dehydrochlorination reactions conducted under the same conditions described above with respect to the dehydrochlorination of the first chloroalkane. For example, the process may further include dehydrochlorinating the second chloroalkane to produce at least some second chloroalkene. The dehydrochlorination of the second chloroalkane may include contacting the second chloroalkane, in liquid phase, with a solid catalyst, a catalyst deposited on a solid substrate, or combinations thereof, thereby causing dehydrochlorination of the second chloroalkane to produce at least some second chloroalkene and HCl. In at least some instances, the dehydrochlorination of the second chloroalkane may include contacting the second chloroalkane with a homogeneous catalyst or a combination of a homogeneous catalyst and a solid catalyst or a catalyst supported on a solid substrate. Further, the chloroalkene product of the second dehydrochlorination reaction, or any other additional dehydrochlorination reaction, may be chlorinated by one or more additional chlorination reactions to form an additional chlorinated alkane product. For example, the second chloroalkene may be chlorinated in the same manner described with reference to the chlorination of the first chloroalkene product to produce a third chloroalkane product or one or more additional chloroalkane products. In at least some instances, the first and second dehydrochlorinations may occur under different reaction conditions. In other cases, the first and second dehydrochlorinations may occur under the same or substantially the same reactions conditions.

The first chloroalkane may be a dichloropropane, trichloropropane, a tetrachloropropane, a pentachloropropane, a hexachloropropane, or combinations thereof. Non-limiting examples of trichloropropanes, tetrachloropropanes, pentachloropropanes, and hexachloropropanes include, but are not limited to 1,1-dichloropropane; 1,2-dichloropropane; 1,3-dichloropropane; 1,1,1-trichloropropane; 1,1,2-trichloropropane; 1,2,2-trichloropropane; 1,2,3-trichloropropane; 1,1,1,2-tetrachloropropane; 1,1,2,2-tetrachloropropane; 1,1,1,3-tetrachloropropane; 1,1,2,3-tetrachloropropane; 1,1,3,3-tetrachloropropane; 1,1,1,2,3-pentachloropropane; 1,1,2,3,3-pentachloropropane; 1,1,2,2,3-pentachloropropane; 1,1,1,3,3-pentachloropropane; 1,1,1,3,3,3-hexachloropropane; 1,1,2,2,3,3-hexachloropropane; or combinations thereof. Generally, the first chloropropanes are at least 90% pure. More preferably, they are at least 95% pure. Still more preferably, they are at least 99% pure.

According to at least one aspect of the present disclosure, the process may include 1,1,1,3-tetrachloropropane and mixtures of 1,1,1,3-tetrachloropropane as the liquid phase first chloroalkane. The first chloroalkene product may include 1,1,3-trichloropropene, 3,3,3-trichloropropene, 1,2,3-trichloropropene, and mixtures thereof. The second chloroalkane may be a pentachloropropane, such as 1,1,1,2,3-pentachloropropane. The second chloroalkene may be a tetrachloropropene, such as 1,1,2,3-tetrachloropropene (1230xa).

Suitable reaction conditions for the presently disclosed process, or portions thereof, include temperatures from about 0° C. to about 200° C. In at least some instances, suitable temperatures may be from about 25° C. to about 170° C. or from about 80° C. to about 150° C. Suitable pressures for the presently disclosed process, or portions thereof, include pressures from about 0 psig to about 200 psig.

According to at least one aspect of the present disclosure, the solid catalyst, catalyst deposited on a solid substrate, or combinations thereof, may be contained in a reactor. In at least some instances, the presently disclosed process, or any portion of the presently disclosed process, may occur in a reactor. In at least some cases, different portions of the presently disclosed process may occur in different reactors. In at least some instances, the reactor may be any suitable liquid phase reactor, such as a batch, semi-batch, or continuous stirred tank reactor. Suitable reactors may include a packed bed reactor, a packed bed column reactor, a continuous stirred tank reactor, a reactive distillation system, a shell and multitube reactor, and combinations thereof.

According to at least one aspect of the present disclosure, the solid catalyst, catalyst deposited on a solid substrate, or combinations thereof, may be contained in a packed bed reactor. In such instances, the presently disclosed process may include feeding the liquid phase first chloroalkane into a packed bed reactor having a first end and a second end. The chloroalkane feed may be fed from either the first end or the second end of the bed of the packed bed reactor. In another example, the process may include a chloroalkane feed into a packed bed column reactor from either the top of the column or the bottom of the column. In other instances, the solid catalyst, catalyst deposited on a solid substrate, or combinations thereof, may be contained in a stirred tank reactor.

According to at least one aspect of the present disclosure, the process may further include removing at least a portion of the first chloroalkene product as a gas or vapor. In at least some instances, the pressure of the reactor may be controlled such that at least some of the first chloroalkene product is caused to flash from the liquid phase to a vapor and exit the reactor as a vapor. In at least some instances, the dehydrochlorination reaction may occur in a reactive distillation system such that at least some of the first chloroalkene product is removed as a vapor and at least some of the reaction liquid comprising the first chloroalkane and heavy by-products is removed as a liquid. In at least some instances, the process may include removing chloroalkene products as they are formed, using a reactive distillation system, thereby reducing catalyst fouling and deactivation.

According to at least one aspect of the present disclosure, the presently disclosed process may be carried out in a shell and multitube reactor capable of near-isothermal operation. In at least some instances, the shell and multi-tube reactor may include a shell having a diameter of several meters that contains from as few as about 5000 tubes up to as many as about 50,000 reaction tubes. Each reaction tube may be as long as 5, 10, or even 15 meters. During typical operation of the shell and multi-tube reactor, desired reactant gases or liquids are supplied to the inlet chamber at the upper ends of the reactor tubes and passed therethrough. Effluents leaving the lower ends of the reactor tubes are collected in the effluent collecting head. The heat of reaction is removed by a heat transfer fluid which is passed across the outer surfaces of the reactor tubes.

In at least some instances, the presently disclosed process may occur in a shell and multitube reactor configured to maintain the temperature during the dehydrochlorination reaction. In such instances, the solid catalyst, catalyst deposited on a solid substrate, or combinations thereof, may be contained in one or more of the tubes in the reactor. Near isothermal conditions may be maintained during the dehydrochlorination reaction using a heat transfer fluid that may include, but is not limited to, heated oil or steam.

According to at least one aspect of the present disclosure, a process for the production of 1,1,1,2,3-pentachloropropane is provided. The process includes contacting 1,1,1,3-tetrachloropropane in liquid phase with a solid catalyst, a catalyst deposited on a solid substrate, or combinations thereof, thereby causing dehydrochlorination of the 1,1,1,3-tetrachloropropane to produce at least one trichloropropene product and HCl. The process further includes reacting at least a portion of the trichloropropene product with a chlorinating agent to form at least some 1,1,1,2,3-pentachloropropane. In at least some instances, the trichloropropene product may include 1,1,3-trichloropropene, 3,3,3-trichloropropene, 1,2,3-trichloropropene, and mixtures thereof. Suitable solid catalysts or catalysts deposited on a solid substrate are the same as those previously described with respect to the dehydrochlorination of the first chloroalkane.

According to at least one aspect of the present disclosure, a process for the production of 1,1,2,3-tetrachloropropene (1230xa) is provided. The process includes contacting 1,1,1,3-tetrachloropropane in liquid phase with a solid catalyst, a catalyst deposited on a solid substrate, or combinations thereof, thereby causing dehydrochlorination of the 1,1,1,3-tetrachloropropane to produce at least one trichloropropene product and HCl. The process further includes reacting at least a portion of the trichloropropene product with a chlorinating agent to form at least some 1,1,1,2,3-pentachloropropane. The process further includes contacting the 1,1,1,2,3-pentachloropropane in liquid phase with a homogeneous catalyst (e.g., metal chlorides of iron, aluminum, tin and/or antimony), a solid catalyst, a catalyst deposited on a solid substrate, or combinations thereof, thereby causing dehydrochlorination of the 1,1,1,2,3-pentachloropropane to produce at least some 1,1,2,3-tetrachloropropene. In at least some instances, the trichloropropene product may include 1,1,3-trichloropropene, 3,3,3-trichloropropene, 1,2,3-trichloropropene, and mixtures thereof. Suitable solid catalysts or catalysts deposited on a solid substrate are the same as those previously described with respect to the dehydrochlorination of the first chloroalkane.

According to at least one aspect of the present disclosure, the process may further include distilling or otherwise purifying one or more of the chloroalkene products or chloroalkane products after such products are removed from the reactor. For example, one or more of the chloroalkene or chloroalkane products may be purified by stripping. In at least some instances, a stripping gas or vapor may be used to strip the chloropropene products from the higher-boiling chloropropane starting material. In such cases, a stripping gas or vapor may be introduced to the reactor or reactive distillation system. In at least some instances, the stripping gas may be HCl gas that is produced as a byproduct of the reaction.

In another aspect of the present disclosure, the process may further include recycling one or more of the chloroalkane products or chloroalkene products after distilling or purifying back into the process. By recycling one or more chloroalkane products and/or chloroalkene products, increased efficiency, increased through-put, and a lower unit manufacturing cost can be attained.

In at least some instances, the presently disclosed process may cause the conversion of between about 10% and about 100% of the first or second chloroalkane to chloroalkene product. According to at least one aspect of the present disclosure, the percent (%) conversion of first or second chloroalkane to chloroalkene product achieved according to the presently disclosed process may be in the range of a lower limit of about 10%, 15%, 20%, 25%, 30%, 35%, 40%, and 50%, to an upper limit of about 55%, 60%, 65%, 70%, 75%, 80%, 85%, 90%, 95% and 100%, encompassing any value and subset therebetween. According to at least one aspect of the present disclosure, the percent (%) conversion of first or second chloroalkane to chloroalkene product achieved according to the presently disclosed process may be in the range of a lower limit of about 10%, 15%, and 20%, to an upper limit of about 25%, 30%, and 35%, encompassing any value and subset therebetween.

In at least some instances, the presently disclosed process is conducted under reaction conditions sufficient to achieve at least 90% conversion of the first or second chloroalkane to chloroalkene product. In other cases, the presently disclosed process may be conducted under reaction conditions sufficient to achieve at least 85% conversion of the first or second chloroalkane to chloroalkene product. In yet other instances, the process may be conducted under reaction conditions sufficient to achieve at least 75% conversion of the first or second chloroalkane to chloroalkene product.

The presently disclosed process, including dehydrochlorination of a chloroalkane to produce a chloroalkene by contacting a liquid phase chloroalkane with a solid catalyst, a catalyst deposited on a solid substrate, or combinations thereof, provides advantages over conventional dehydrochlorination processes that use dissolved catalysts, such as dissolved metal chlorides. The use of a solid catalyst or a catalyst deposited on a solid substrate obviates the need to distill or otherwise separate the chloroalkene products from the dissolved catalyst. The elimination of the need for dissolved catalytic components has the additional advantage of preventing further undesired catalytic reactions that may occur prior to or during the distillation or separation process.

In an additional aspect, disclosed herein are processes for the conversion of the chloroalkenes, such as 1,1,2,3-tetrachloropropane, and/or the chloroalkanes, such as 1,1,1,2,3-pentachloropropane, to one or more hydrofluoroolefins. These processes comprise contacting the chloroalkene and/or chloroalkane with a fluorinating agent in the presence of a fluorination catalyst, in a single reaction or two or more reactions. These processes can be conducted in either gas phase or liquid phase with the gas phase being preferred at temperatures ranging from 50° C. to 400° C.

Generally, a wide variety of fluorinating agents can be used. Non-limiting examples of fluorinating agents include HF, F₂, ClF, AlF₃, KF, NaF, SbF₃, SbF₅, SF₄, or combinations thereof. The skilled artisan can readily determine the appropriate fluorination agent and catalyst. Examples of hydrofluoroolefins that may be produced utilizing these processes include, but are not limited to 2,3,3,3-tetrafluoroprop-1-ene (HFO-1234yf), 1,3,3,3-tetrafluoroprop-1-ene (HFO-1234ze), 3,3,3-trifluoroprop-1-ene (HFO-1243zf), and 1-chloro-3,3,3-trifluoroprop-1-ene (HFCO-1233zd).

### Example 1

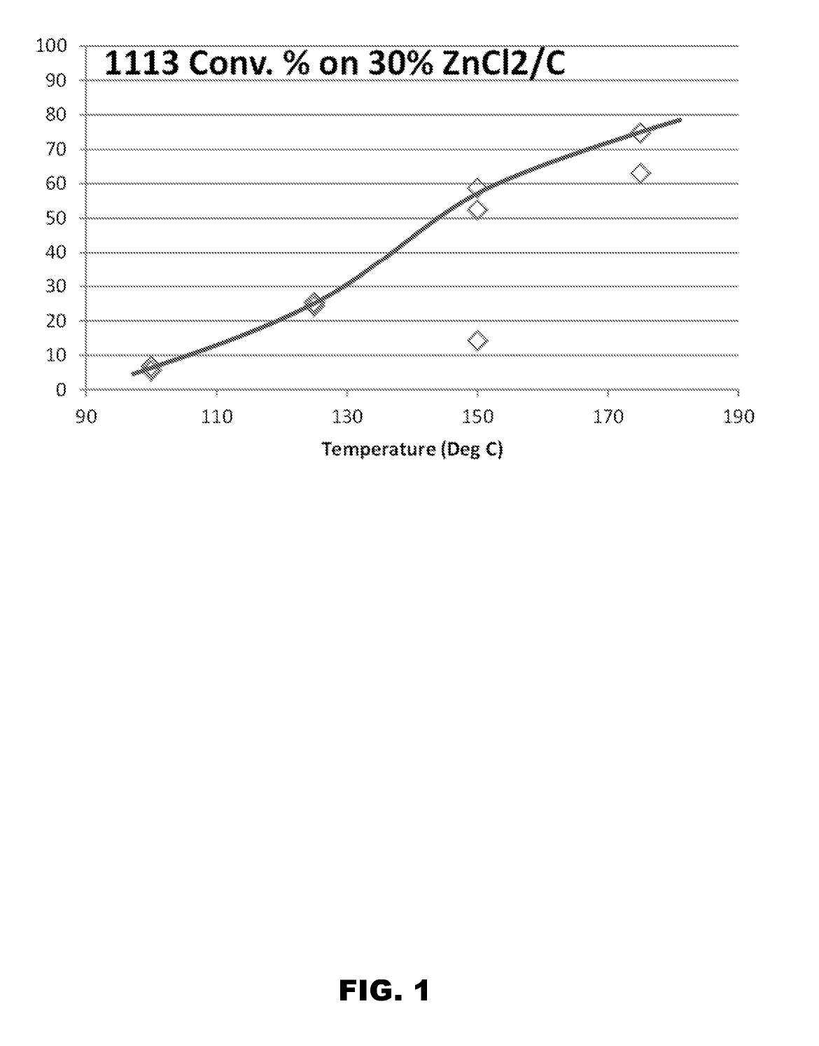

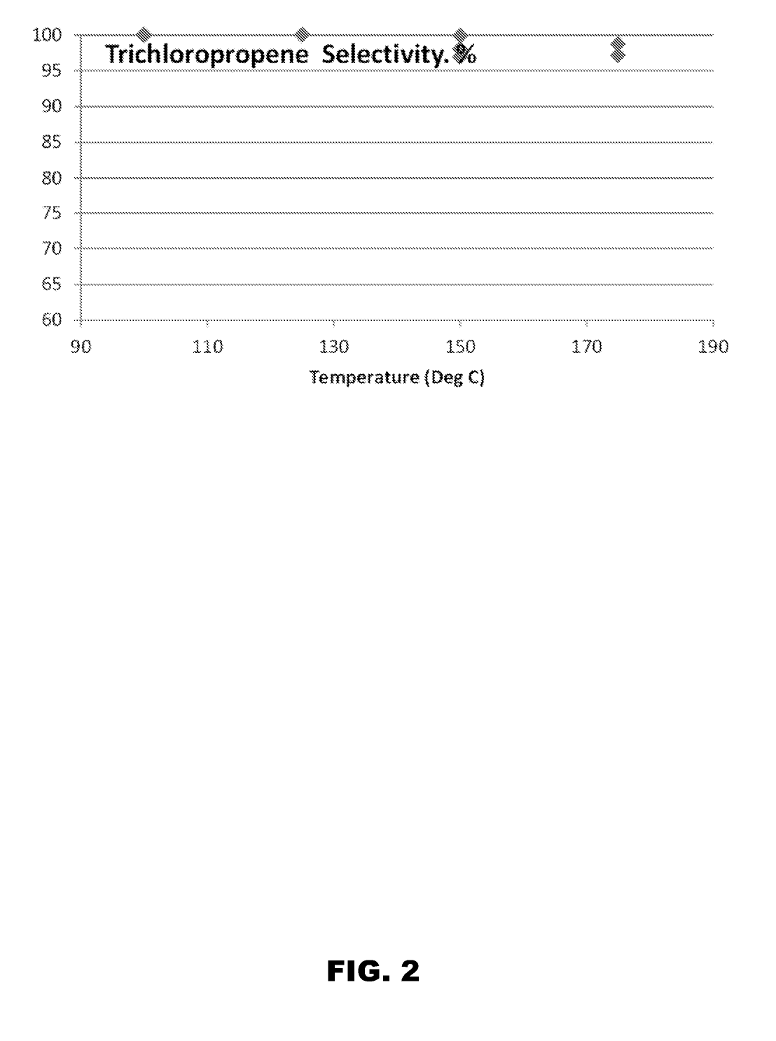

A catalyst comprising 5.6 g of 30 weight percent ZnCl₂ deposited on a carbon solid substrate was charged to a 25.4 cm long Monel tube having a 0.77 cm internal diameter. Both ends of the Monel tube were fitted with screens to hold the catalyst inside the tube. The catalyst was purged with nitrogen for 2 hours at 200° C. The temperature was reduced to 100° C. and liquid phase 1,1,1,3-tetrachloropropane was fed to the Monel tube at a flow rate of 0.33 cc/min. Pressure was controlled at 170 psig. Temperature was varied and samples of the liquid exiting the tube were collected at different temperatures and analyzed by gas chromatography. FIG. 1 shows the percentage conversion of 1,1,1,3-tetrachloropropane versus temperature while FIG. 2 shows the percentage trichloropropene selectivity versus temperature.

### Example 2

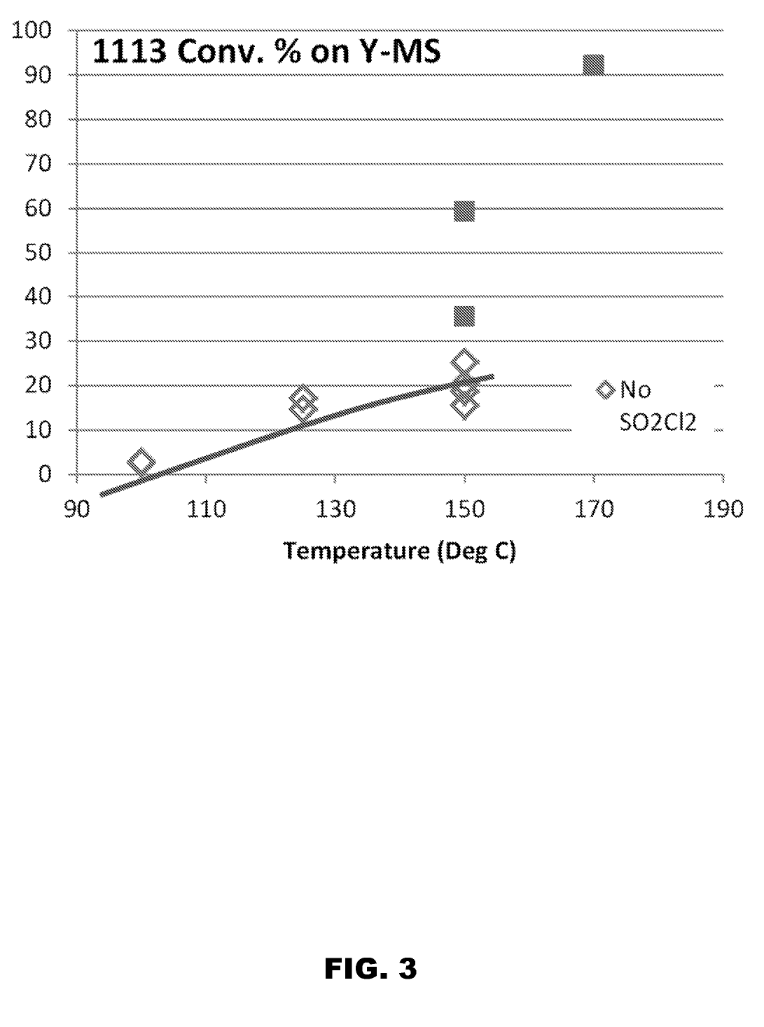

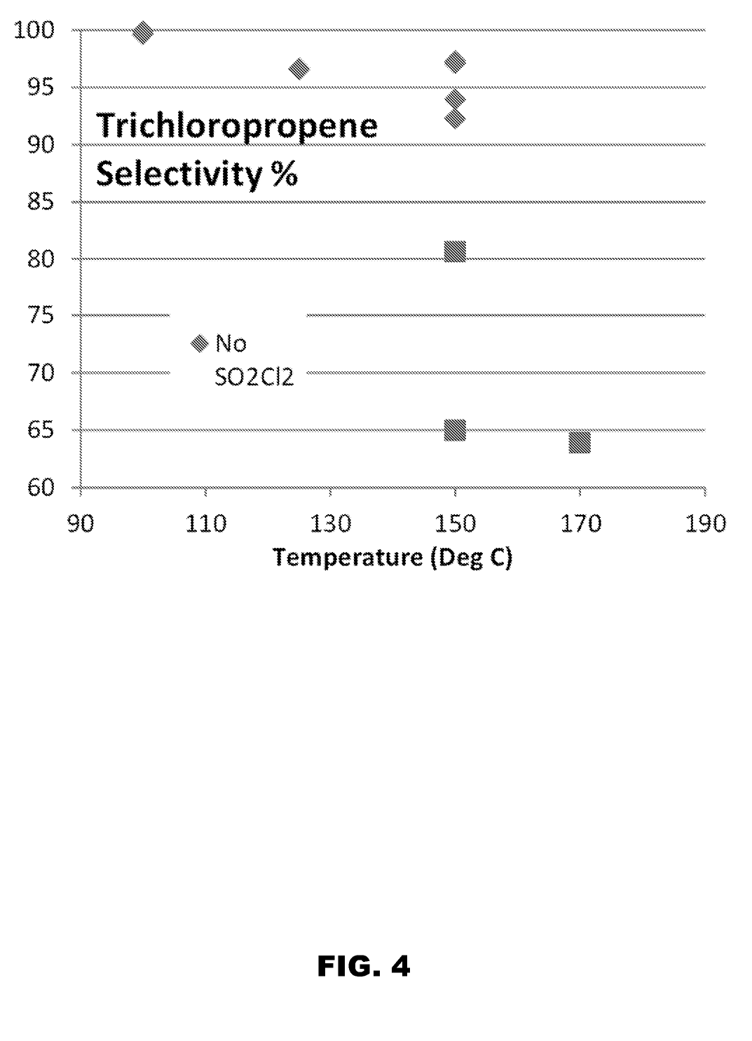

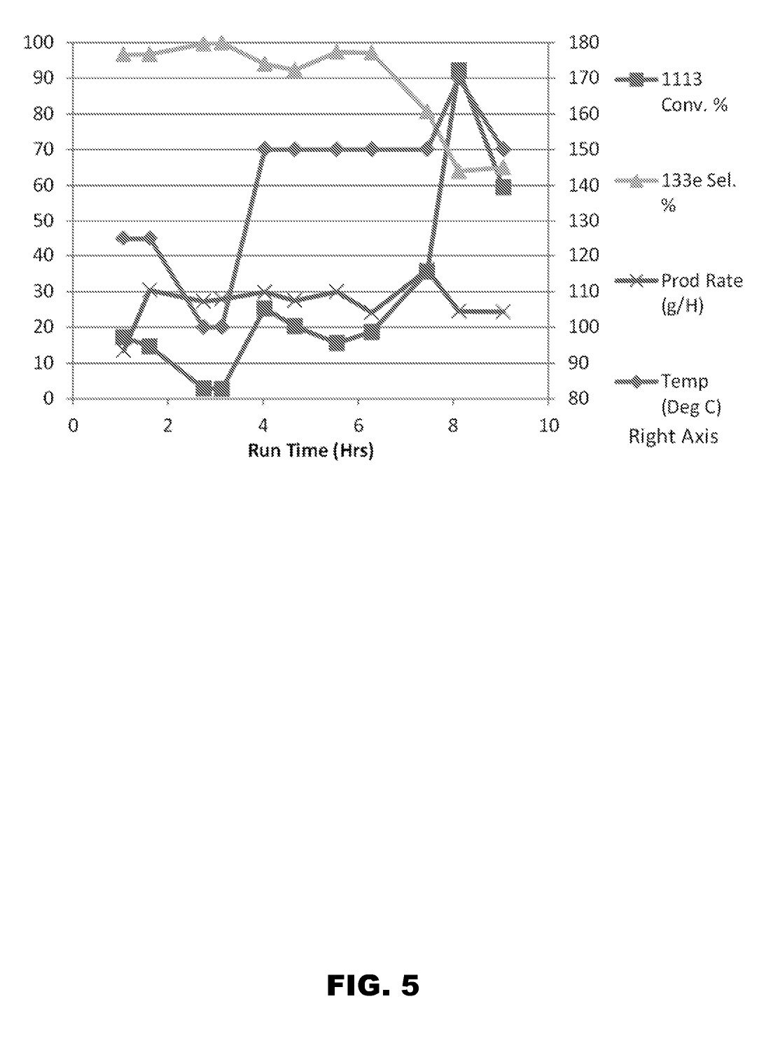

A catalyst comprising 5 g y-type molecular sieve was charged to a 25.4 cm long Monel tube having a 0.77 cm internal diameter. Both ends of the Monel tube were fitted with screens to hold the catalyst inside the tube. The catalyst was purged with nitrogen for 2 hours at 200° C. The temperature was reduced to 100° C. and liquid phase 1,1,1,3-tetrachloropropane was fed to the Monel tube at a flow rate of 0.33 cc/min. Pressure was controlled at 170 psig. Temperature was varied and samples of the liquid exiting the tube were collected at different temperatures and analyzed by gas chromatography. Sulfuryl chloride was added to the feed to provide simultaneous chlorination of trichloropropene as it was formed. Samples of the liquid exiting the tube were collected at different temperatures and analyzed by gas chromatography. FIG. 3 shows the percentage conversion of 1,1,1,3-tetrachloropropane versus temperature while FIG. 4 shows the percentage trichloropropene selectivity versus temperature, with and without simultaneous chlorination of the trichloropropene product by sulfuryl chloride. FIG. 5 shows the percent conversion of 1,1,1,3-tetrachloropropane, percent trichloropropene selectivity, and production rate in g/H versus time (percentage (%) or production rate (g/H) is on the left y-axis, and temperature is on the right y-axis). As shown in FIG. 3, conversion of 1,1,1,3-tetrachloropropane increased when chlorinating agent was added to the chloroalkane feed. As shown in FIGS. 3 and 5, the four data points at 150° C. prior to the addition of sulfuryl chloride to the feed, shows gradually declining conversion with time during the continuous feed corresponding to gradual catalyst deactivation.

### Example 3

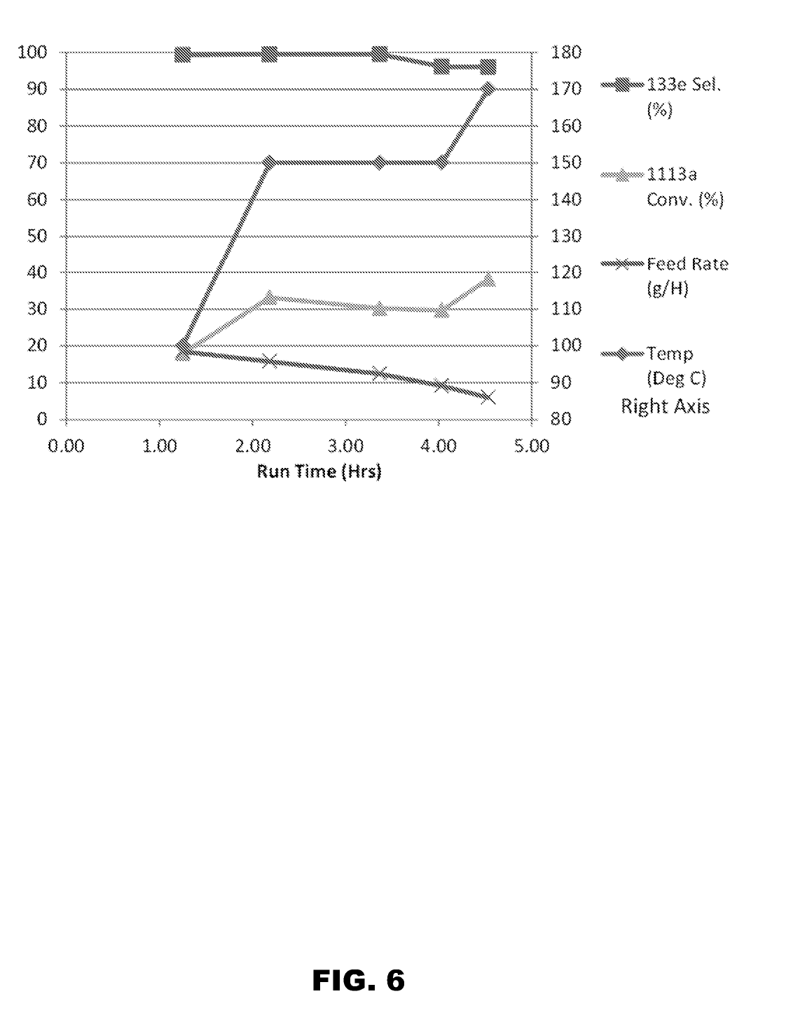

A catalyst comprising 4.9 g 13× molecular sieve in the form of 16×40 mesh beads was charged to a 25.4 cm long Monel tube having a 0.77 cm internal diameter. Both ends of the Monel tube were fitted with screens to hold the catalyst inside the tube. The catalyst was purged with nitrogen for 2 hours at 200° C. The temperature was reduced to 100° C. and liquid phase 1,1,1,3-tetrachloropropane was fed to the Monel tube at a flow rate of 0.33 cc/min. Pressure was controlled at 170 psig. Temperature was varied and samples of the liquid exiting the tube were collected at different temperatures and analyzed by gas chromatography. FIG. 6 shows the percentage conversion of 1,1,1,3-tetrachloropropane, percentage trichloropropene selectivity, and feed rate in g/H versus time (percentage (%) or feed rate (g/H) is on the left y-axis, and temperature is on the right y-axis). As shown in FIG. 6, the percent (%) conversion decreased over time indicating gradual catalyst deactivation during the continuous feed. Deactivation of the catalyst over time would have been even more pronounced if the flow rate had not declined during the course of the continuous feed. Deactivation of the catalyst over time indicates the potential benefit of removing at least some of the chloroalkene products from the liquid as they are formed in order to prevent coke formation leading to catalyst deactivation.

### Example 4

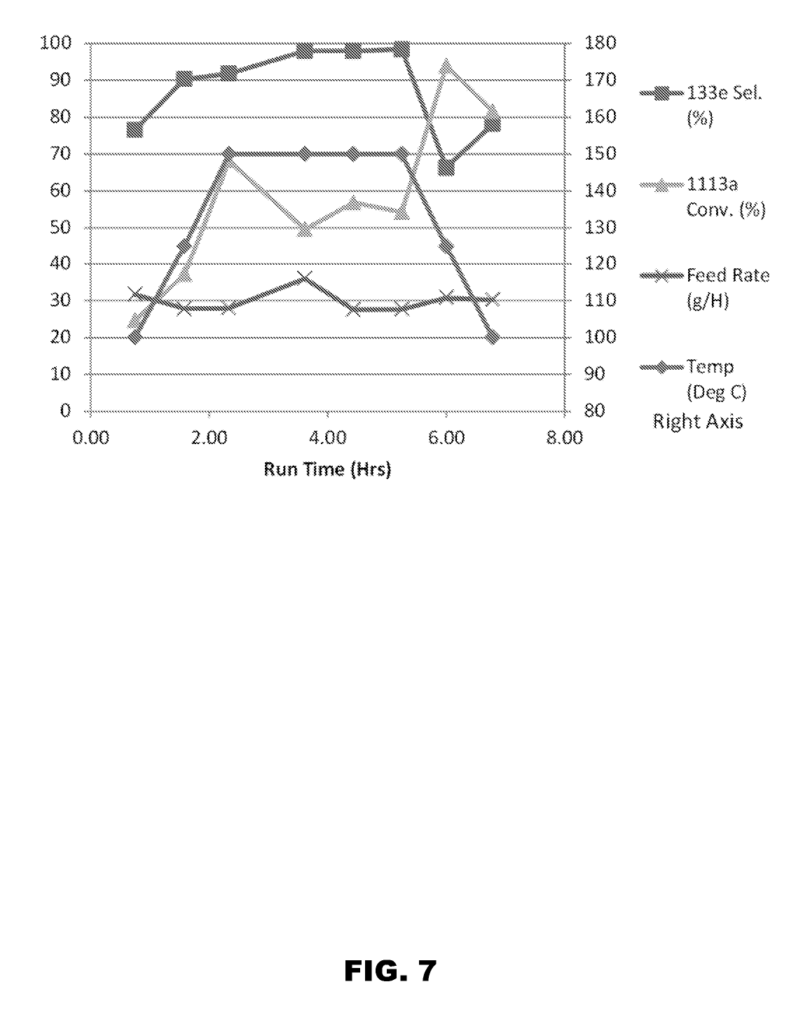

A catalyst comprising 4.2 g 22.9 weight percent FeCl₃ on activated carbon was charged to a 25.4 cm long Monel tube having a 0.77 cm internal diameter. Both ends of the Monel tube were fitted with screens to hold the catalyst inside the tube. The catalyst was purged with nitrogen for 1 hour at 200° C. The temperature was reduced to 100° C. and liquid phase 1,1,1,3-tetrachloropropane was fed to the Monel tube at a flow rate of 0.33 cc/min. Pressure was controlled at 100 psig. Temperature was varied and samples of the liquid exiting the tube were collected at different temperatures and analyzed by gas chromatography. FIG. 7 shows the percentage conversion of 1,1,1,3-tetrachloropropane, percentage trichloropropene selectivity, and feed rate in g/H versus time (percentage (%) or feed rate (g/H) is on the left y-axis, and temperature is on the right y-axis). As shown in FIG. 7, the percent (%) conversion decreased over time indicating gradual catalyst deactivation during the continuous feed. Deactivation of the catalyst over time indicates the potential benefit of removing at least some of the chloroalkene products from the liquid as they are formed in order to prevent coke formation leading to catalyst deactivation. Sulfuryl chloride was added to the feed in the amount of 37 percent by weight. The last two data points in FIG. 7 with sulfuryl chloride in the feed show increased conversion and lower selectivity to 1,1,3-trichloropropene as a result of some 1,1,3-trichloropropene being chlorinated to 1,1,1,2,3-pentachloropropane.

Although a variety of examples were used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples or process steps, it is to be understood that the subject matter defined in the appended claims is not necessarily limited to these described features or acts. Rather, the described features and processes are disclosed as examples of components of systems and methods within the scope of the appended claims.

1. A process for the production of a chloroalkene comprising:
contacting a liquid phase first chloroalkane with a solid catalyst, a catalyst deposited on a solid substrate, or combinations thereof, and thereby causing dehydrochlorination of the first chloroalkane to produce a first chloroalkene product and HCl.

2. The process according to claim 1, wherein the catalyst comprises a metal chloride deposited on a substrate comprising carbon, alumina, silica, titania, an aluminosilicate, a zeolite, or combinations thereof.

3. The process according to claim 1, wherein the catalyst comprises a reduced (zero-valence) metal deposited on a substrate comprising carbon, alumina, silica, titania, an aluminosilicate, a zeolite, or combinations thereof.

4. The process according to claim 1, wherein the first chloroalkane comprises a chloropropane.

5. The process according to claim 1, wherein the first chloroalkane comprises 1,1,1,3-tetrachloropropane and the first chloroalkene product comprises 1,1,3-trichloropropene, 3,3,3-trichloropropene, 1,2,3-trichloropropene, or mixtures thereof.

6. The process according to claim 1, wherein the catalyst comprises ZnCl₂, FeCl₃, CuCl₂, or combinations thereof, deposited on a carbon substrate.

7. The process according to claim 1, wherein the solid catalyst, a catalyst deposited on a solid substrate, or combinations thereof, comprises an aluminosilicate molecular sieve, a zeolite molecular sieve, a Y-type molecular sieve, a 13× molecular sieve, or combinations thereof.

8. The process according to claim 1, further comprising removing at least a portion of the first chloroalkene product as a gas or vapor.

9. The process according to claim 1, further comprising reacting at least a portion of the first chloroalkene product with a chlorinating agent to form at least some of a second chloroalkane product, wherein the first chloroalkane and the second chloroalkane are different.

10. The process according to claim 9, wherein the chlorinating agent comprises chlorine gas, liquid chlorine, sulfuryl chloride, or combinations thereof.

11. The process according to claim 9, further comprising dehydrochlorinating the second chloroalkane to produce at least some second chloroalkene.

12. (canceled)

13. The process according to claim 9, wherein the second chloroalkane comprises a pentachloropropane.

14. The process according to claim 11, wherein the second chloroalkene comprises a tetrachloropropene.

15. The process according to claim 13, wherein the pentachloropropane is 1,1,1,2,3-pentachloropropane.

16. The process according to claim 14, wherein the tetrachloropropene is 1,1,2,3-tetrachloropropene.

17. The process according to claim 1, wherein the dehydrochlorination of the first chloroalkane and the chlorination of the first chloroalkene product occurs contemporaneously in the same vessel, in different sections of the same vessel, or in separate vessels.

18. The process according to claim 1, wherein dehydrochlorination of the first chloroalkane or second chloroalkane is conducted at a temperature of from about 0° C. to about 200° C. and at a pressure of from about 0 psig to about 200 psig.

19. The process according to claim 1, wherein at least a portion of the process is conducted in a reactor comprising a packed bed reactor, a packed bed column reactor, a continuous stirred tank reactor, a reactive distillation system, a shell and multitube reactor, or combinations thereof.

20. (canceled)

21. (canceled)

22. The process according to claim 1, further comprising removing at least some of the first chloroalkene product as a vapor and removing at least some of the first chloroalkane as a liquid.

23. The process according to claim 11, wherein the dehydrochlorination of the first chloroalkane or the second chloroalkane occurs in a reactive distillation system, the process further comprising removing at least some of the first chloroalkene product as a vapor and removing at least some of the first chloroalkane or the second chloroalkane as a liquid.

24. The process according to claim 11, wherein the solid catalyst, catalyst deposited on a solid substrate, or combinations thereof, is contained in at least a plurality of reactor tubes of a shell and multitube reactor, the process further comprising maintaining the dehydrochlorination of the first chloroalkane or the second chloroalkane at near-isothermal operating conditions by passing a heat transfer fluid across the outer surfaces of the reactor tubes.

25. (canceled)

26. A process for the production of 1,1,1,2,3-pentachloropropane comprising:
contacting 1,1,1,3-tetrachloropropane in liquid phase with a solid catalyst, a catalyst deposited on a solid substrate, or combinations thereof, and thereby causing dehydrochlorination of the 1,1,1,3-tetrachloropropane to produce a trichloropropene product and HCl; and
reacting at least a portion of the trichloropropene product with a chlorinating agent to form at least some 1,1,1,2,3-pentachloropropane.

27. A process for the production of 1,1,2,3-tetrachloropropene comprising:
contacting 1,1,1,3-tetrachloropropane in liquid phase with a solid catalyst, a catalyst deposited on a solid substrate, or combinations thereof, and thereby causing dehydrochlorination of the 1,1,1,3-tetrachloropropane to produce a trichloropropene product and HCl;
reacting at least a portion of the trichloropropene product with a chlorinating agent to form at least some 1,1,1,2,3-pentachloropropane; and
contacting the 1,1,1,2,3-pentachloropropane in liquid phase with a homogeneous catalyst, a solid catalyst, a catalyst deposited on a solid substrate, or combinations thereof, and thereby causing dehydrochlorination of the 1,1,1,2,3-pentachloropropane to produce at least some 1,1,2,3-tetrachloropropene.

28. (canceled)

29. The process of claim 1, wherein the chloroalkene and/or the chloroalkane is converted to produce a fluorinated product.
